(19) United States
(12) Patent Application Publication
(10) Pub. No.: US 2016/0348181 A1
(43) Pub. Date: Dec. 1, 2016

(54) QUANTITATIVE REVERSE TRANSCRIPTION POLYMERASE CHAIN REACTION KIT USING TISSUE AND BLOOD FOR EARLY DIAGNOSIS AND SCREENING TEST FOR THERAPEUTIC AGENT OF BREAST CANCER

(71) Applicants: OPTIPHARM. CO., LTD., Cheongju-si (KR); UNIVERSITY INDUSTRY FOUNDATION, YONSEI UNIVERSITY WONJU CAMPUS, Wonju-si (KR)

(72) Inventors: Hye Young LEE, Wonju-si (KR); Hye Young WANG, Gunsan-si (KR); Yeun KIM, Wonju-si (KR)

(21) Appl. No.: 15/104,592

(22) PCT Filed: Dec. 20, 2013
(86) PCT No.: PCT/KR2013/011978

§ 371 Date: Jun. 15, 2016

(30) Foreign Application Priority Data
Jan. 7, 2014 (JP) 2014-001074

(51) Int. Cl. C12Q 1/68

(52) U.S. Cl. CPC C12Q 1/6886; C12Q 2600/158; C12Q 2600/16

(57) ABSTRACT

There are provided a method for an early diagnosis and a screening test for a therapeutic agent of breast cancer by using tissue and blood, and a quantitative reverse transcription polymerase chain reaction kit for same. According to the present invention, it is possible to provide help in more effective treatment and diagnosis of breast cancer through expression rates of HER2 expressed in blood and a cancer-related marker in the blood in addition to a tissue specimen.

### TECHNICAL FIELD

The present invention relates to a method for an early diagnosis and a screening test for a therapeutic agent of breast cancer by using tissue and blood, and a quantitative reverse transcription polymerase chain reaction kit for same.

### BACKGROUND ART

Human epidermal growth factor receptor 2 (HER2) is an ErbB-like oncogene family and a HER2 test is very important in treatment for breast cancer. Particularly, overexpression of the HER2 is detected in 20 to 30% of breast cancer patients, and in 20 to 30% of the patients, prognosis is not good, a more severe aspect of the breast cancer is shown, and a 5-year survival rate is reduced as compared with the remaining patients. For the treatment of the HER2 overexpression patients, Herceptin (Roche) is used. The Herceptin is a humanized monoclonal antibody and directly targets an extracellular domain of the HER2 receptor. Currently, the Herceptin has been used as chemotherapy in treatment of metastatic breast cancer patients and treatment of neo-adjuvant patients.

In a diagnosis of cancer patients, fluorescence in situ hybridization (FISH) and immunohistochemistry (IHC) have been used as at the most gold standard in stages, prognosis, or therapeutic agent screening of the cancer. Particularly, in an overexpression diagnosis of the HER2, the IHC method is used as a method representing whether a HER2 protein is overexpressed on the surface of cancer cells, and the FISH method is used as a method of finding whether a HER2 gene on a chromosome is overexpressed. Particularly, the IHC method is the most common method as a primary screening test, but there is a different for each test agency, and technical accuracy or reproduction of the results is discussed. Further, it is known that the FISH method is better than the IHC method in a matching rate of the result, and better than the IHC method in sensitivity and specificity. However, it is known that since the test process is very complicated and a test method using fluorescence, permanent preservation of the result is impossible. Further, it is known that since a price of a fluorescent probe is very expensive, the performance of the test process is impossible in small hospitals and the like.

As related prior patent documents, Korean Patent Publication No. 1020090079845 relates to ‘a protein marker for monitoring, diagnosing, and screening breast cancer and a method for monitoring, diagnosing, and screening breast cancer using the same’ and Korean Patent Publication No. 1020090064378 relates to ‘genes and polypeptides relating to breast cancers’.

### DISCLOSURE

### Technical Problem

In order to solve the conventional problems, an object of the present invention is to provide an information providing method for a diagnosis of breast cancer using quantitative reverse transcription polymerase chain reaction.

Another object of the present invention is to provide a kit for a diagnosis of breast cancer.

### Technical Solution

In order to achieve the above object, an exemplary embodiment of the present invention provides an information providing method for diagnosing breast cancer, comprising: a) isolating a total RNA from cells obtained from tissue or blood of a cancer suspected patient; b) synthesizing cDNA from the isolated total RNA; c) performing real-time-PCR of the synthesized cDNA by using compositions of a primer set and a probe to amplify a human epidermal growth factor receptor (HER) 2, a primer set and a probe to amplify an estrogen receptor, a primer set and a probe to amplify a progesterone receptor, a primer set and a probe to amplify cytochrome keratin 19, a primer set and a probe to amplify an epithelial cell adhesion molecule (EpCAM), a primer set and a probe to amplify human telomerase reverse transcriptase (hTERT), a primer set and a probe to amplify Ki67, a primer set and a probe to amplify Vimentin, a primer set and a probe to amplify cyclin D1, a primer set and a probe to amplify E-cadherin (cad), a primer set and a probe to amplify snail, a primer set and a probe to amplify phosphatase and tensin homolog (PTEN), a primer set and a probe to amplify neuroplastin (NPTN), and a primer set and a probe to amplify glyceraldehyde-3-phosphate dehydrogenase (GAPDH); and d) comparing the amplified level with an expressed level in a normal person.

The method of separating the total RNA and the method of synthesizing cDNA from the separated total RNA which are generally used may be performed through known methods, and the detailed description for the process is described in Joseph Sambrook et al., Molecular Cloning, A Laboratory Manual, Cold Spring Harbor Laboratory Press, Cold Spring Harbor, N.Y. (2001); Noonan, K. F., and the like and may be inserted as the reference of the present invention.

The primer of the present invention may be chemically synthesized b using a phosphoramidite solid support method or other well-known methods. The nucleic acid sequence may be modified by using many means known in the art. As an unlimited example of the modification, there are methylation, “capsulation”, substitution to analogues of one or more nucleotides, and modification between nucleotides, for example, modification to a non-charged connection body (For example, methyl phosphonate, phosphotriester, phosphoroamidate, carbamates, and the like) or a charged connection body (for example, phosphorothioate, phosphorodithioate, and the like). The nucleic acid may contain one or more additional covalently-linked residues, for example, a protein (for example, nucleases, toxins, antibodies, signal peptides, poly-L-lysine, and the like), an intercalating agent (for example, acridine, psoralen, and the like), a chelating agent (for example, metal, radioactive metal, iron, oxidative metal, and the like), and an alkylating agent. The nucleic acid sequence of the present invention may be modified by using a marker which may directly or indirectly provide a detectable signal. An example of the marker includes a radioisotope, a fluorescent molecule, biotin, or the like.

In the method of the present invention, the amplified target sequence (HER 2, GAPDH genes, and the like) may be marked by a detectable marking material. In an exemplary embodiment, the marking material may be a fluorescent, phosphorescent, chemiluminescent, or radioactive material, but the present invention is not limited thereto. Preferably, the marking material may be fluorescein, phycoerythrin, rhodamine, lissamine, Cy-5 or Cy-3. When RT-PCR is performed by marking Cy-5 or Cy-3 in a 5′-terminal and/or a 3′-terminal of the primer when amplifying the target sequence, the target sequence may be marked with the detectable fluorescent marking material.

Further, in the marker using the radioactive material, when the radioactive isotope such as ³²P or ³⁵S during the RT-PCR is added in a PCR reaction solution, the amplified production is synthesized and the radioactive material is inserted to the amplified product, and thus, the amplified product may be marked with the radioactive material. One or more oligonucleotide primer sets used for amplifying the target sequence may be used.

The marking may be performed by various methods which are generally performed in the art, for example, a nick translation method, a random priming method (Multiprime DNA labelling systems booklet, “Amersham” (1989)), and a keynation method (Maxam & Gilbert, Methods in Enzymology, 65:499(1986)). The marking provides a signal which is detectable by fluorescence, radioactivity, color measurement, weight measurement, X-ray diffraction or absorption, magnetism, enzymatic activity, mass analysis, binding affinity, hybridization high frequency, and nanocrystalline.

According to an aspect of the present invention, in the present invention, an expression level at an mRNA level is measured through RT-PCR. To this end, a new primer set which is specifically bound to the HER 2 and GAPDH genes and a probe marked with fluorescence are required, and in the present invention, the corresponding primer and probe specified with specific base sequences may be used, but the present invention is not limited thereto. The primer and probe may be used without limitation so long as performing the RT-PCR by providing a detectable signal which is specifically bound to the genes. Herein, FAM and Quen (Quencher) mean fluorescent dyes.

The RT-PCR method applied to the present invention may be performed through a known process which is generally used in the art.

The process of measuring the mRNA expression level may be used with limitation as long as measuring the mRNA expression level and may be performed through radiation measurement, fluorescence measurement or phosphorescence measurement according to a kind of used probe marker, but the present invention is not limited thereto. As one of the method of detecting the amplified product, in the phosphorescence measurement method, when the real-time RT-PCR is performed by marking Cy-5 or Cy-3 in the 5′-terminal of the primer, the target sequence is marked with a detectable fluorescent marker, and the marked fluorescence may be measured by using a fluorescence meter. Further, in the radioactive measurement method, when the RT-PCR is performed, after the amplified product is marked by adding the radioisotope such as ³²P or ³⁵S in a PCR reaction solution, the radioactive material may be measured by using radiation measuring equipment, for example, a Geiger counter or a liquid scintillation counter.

According to an exemplary embodiment of the present invention, the probe marked with the fluorescence is attached to the PCR product amplified through the RT-PCR to emit fluorescence having a specific wavelength. Simultaneously with amplification, in the fluorescence meter of the PCR device, the mRNA expression level of the genes of the present invention is measured in real time, the measured value is calculated and visualized through PC and thus, a checker may easily check the expression level.

According to another aspect of the present invention, the diagnosis kit may be a kit for a breast cancer diagnosis comprising a required element required for performing a reverse transcription polymerase reaction. The reverse transcription polymerase reaction kit may include each gene-specific primer set of the present invention. The primer is a nucleotide having a specific sequence to a nucleic acid sequence of each marker gene and may have a length of approximately 7 bp to 50 bp, and more preferably a length of approximately 10 bp to 30 bp.

Other reverse transcription polymerase reaction kits may include a test tube or another suitable container, a reaction buffer (pH and magnesium concentration are varied), deoxynucleotide (dNTPs), enzymes such as Taq-polymerase and reverse transcriptase, DNAse, RNAse inhibitor, DEPC-water, sterile water, and the like.

In the present invention, the term “information providing method for cancer diagnosis” is to provide objective basic information required for the diagnosis of cancer as a preliminary step and clinical determination or opinions of doctors are excluded.

The term “primer” means a short nucleic acid sequence which may form a complementary template and a base pair as a nucleic acid sequence having a short free 3-terminal hydroxyl group and serves as a starting point for duplicating the template strand. The primer may initiate DNA synthesis under a presence of a reagent for polymerization (that is, DNA polymerase or reverse transcriptase) at a temperature and four different nucleoside triphosphates in a suitable buffer solution. The primer of the present invention is sense or antisense nucleic acid having 7 to 50 nucleotide sequences as a marker gene-specific primer. The primer may combine an additional feature without changing a basic property of the primer acting as an initial point of the DNA synthesis.

The term “probe” is a single chain nucleic acid molecule and includes a complementary sequence to a target nucleic acid sequence.

The term ‘real-time RT-PCR’ is a molecular polymerization method of reverse-transcribing RNA to complementary DNA (cDNA) by using a reverse transcriptase, amplifying a target by using a target probe including a target primer and a marker by using the made cDNA as a template, and simultaneously, quantitatively detecting a signal generated in the marker of the target probe in the amplified target.

According to another exemplary embodiment of the present invention, the primer set to amplify the HER 2 may be transcribed in SEQ ID NOS: 1 to 2, 3 to 4, and 6 to 7 and the probe may be transcribed in SEQ ID NOS: 5, 8, and 9, the primer set to amplify the estrogen receptor may be transcribed in SEQ ID NOS: 10 to 11 and 13 to 14 and the probe may be transcribed in SEQ ID NOS: 12 and 15, the primer set to amplify the progesterone receptor may be transcribed in SEQ ID NOS: 16 to 17 and 19 to 20 and the probe may be transcribed in SEQ ID NOS: 18 and 21, the primer set and the probe to amplify the GAPDH are transcribed in SEQ ID NOS: 22 to 23 and 24, respectively, the primer set and the probe to amplify the EpCAM are transcribed in SEQ ID NOS: 25 to 26 and 27, respectively, the primer set and the probe to amplify the cytochrome keratin 19 may be transcribed in SEQ ID NOS: 28 to 29 and 30, respectively, the primer set and the probe to amplify the hTERT may be transcribed in SEQ ID NOS: 31 to 32 and 33, respectively, the primer set and the probe to amplify the Ki67 may be transcribed in SEQ ID NOS: 34 to 35 and 36, respectively, the primer set and the probe to amplify the Vimentin may be transcribed in SEQ ID NOS: 37 to 38 and 39, the primer set and the probe to amplify the Cyclin D1 may be transcribed in SEQ ID NOS: 40 to 41 and 42, respectively, the primer set and the probe to amplify the E-cad may be transcribed in SEQ ID NOS: 43 to 44 and 45, respectively, the primer set and the probe to amplify the Snail may be transcribed in SEQ ID NOS: 46 to 47 and 48, respectively, the primer set and the probe to amplify the PTEN may be transcribed in SEQ ID NOS: 49 to 50 and 51, respectively, the primer set and the probe to amplify the NPTN may be transcribed in SEQ ID NOS: 52 to 53 and 54, respectively. However, the present invention is not limited thereto.

Another exemplary embodiment of the present invention provides a composition of primer sets and probes for diagnosing breast cancer, comprising: a primer set to amplify HER 2 transcribed in SEQ ID NOS: 1 to 2, 3 to 4, and 6 to 7 and a probe transcribed in SEQ ID NOS: 5, 8, and 9, a primer set to amplify an estrogen receptor transcribed in SEQ ID NOS: 10 to 11 and 13 to 14 and a probe transcribed in SEQ ID NOS: 12 and 15, a primer set to amplify a progesterone receptor transcribed in SEQ ID NOS: 16 to 17 and 19 to 20 and a probe transcribed in SEQ ID NOS: 18 and 21, a primer set and a probe to amplify GAPDH transcribed in SEQ ID NOS: 22 to 23 and 24, respectively, a primer set and a probe to amplify EpCAM transcribed in SEQ ID NOS: 25 to 26 and 27, respectively, a primer set and a probe to amplify cytochrome keratin 19 transcribed in SEQ ID NOS: 28 to 29 and 30, respectively, a primer set and a probe to amplify hTERT transcribed in SEQ ID NOS: 31 to 32 and 33, respectively, a primer set and a probe to amplify Ki67 transcribed in SEQ ID NOS: 34 to 35 and 36, respectively, a primer set and a probe to amplify Vimentin transcribed in SEQ ID NOS: 37 to 38 and 39, a primer set and a probe to amplify Cyclin D1 transcribed in SEQ ID NOS: 40 to 41 and 42, respectively, a primer set and a probe to amplify E-cad transcribed in SEQ ID NOS: 43 to 44 and 45, respectively, a primer set and a probe to amplify Snail transcribed in SEQ ID NOS: 46 to 47 and 48, respectively, a primer set and a probe to amplify PTEN transcribed in SEQ ID NOS: 49 to 50 and 51, respectively, and a primer set and a probe to amplify NPTN transcribed in SEQ ID NOS: 52 to 53 and 54, respectively.

According to an exemplary embodiment of the present invention, a 5′-terminal of the probe may be marked with a fluorescent material, but the present invention is not limited thereto.

Yet another exemplary embodiment of the present invention provides a composition for diagnosing breast cancer including the composition of the primer sets and the probes of claim 4 as an active ingredient.

Still another exemplary embodiment of the present invention provides a kit for diagnosing breast cancer including the composition of the present invention.

Also, the present invention provides a kit for diagnosing breast cancer in an early stage or for each stage including the composition of the present invention.

Hereinafter, the present invention will be described.

In order to replace the part, based on a RT-qPCR method capable of easily deriving quantitative results, expression levels shown after amplifying a HER2 gene using GAPDH as a reference gene are compared and quantified to compare an expression rate with results of IHC and FISH as existing methods. In addition, for effective treatment, the present invention is completed to provide help in more effective treatment and diagnosis of breast cancer through expression rates of HER2 expressed in blood and a cancer-related marker in the blood in addition to a tissue specimen.

### Effect

According to the present invention, it is possible to provide help in more effective treatment and diagnosis of breast cancer through expression rates of HER2 expressed in blood and a cancer-related marker in the blood in addition to a tissue specimen.

### DESCRIPTION OF DRAWINGS

FIG. 1 illustrates verification for HER2 sensitivity of RT-qPCR using a SK-BR-3 cell line and FIG. 2 illustrates verification for improved HER2 sensitivity of one-tube nested RT-qPCR using a SK-BR-3 cell line.

FIG. 3 illustrates an expression rate comparison of HER2 mRNA using a breast cancer cell line.

FIG. 4 illustrates an analysis result of a correlation between HER2 RT-qPCR and HER2 IHC.

FIGS. 5 to 7 illustrate an ROC curve analysis method for clinical Cut-off determination.

FIG. 8 illustrates setting of clinical Cut-off using an ROC curve analysis method.

FIG. 9 illustrates a result of clinical specimens using RT-qPCR.

FIGS. 10 to 12 illustrate a change in sensitivity of hormone receptors using RT-qPCR.

FIGS. 13 and 14 illustrate an ER expression level comparison and an ER-PR mix expression level comparison in clinical specimens using RT-qPCR, respectively.

FIG. 15 illustrates verification of sensitivity of HER2 RT-qPCR after mixing SK-BR-3 with normal blood.

FIG. 16 illustrates a HER2 expression comparison in blood of a normal group and a breast cancer patient group.

FIGS. 17 to 24 illustrate a comparison of expression rates for cancer-related markers in blood.

FIGS. 25 to 28 illustrate a comparison of whether cancer-related markers are expressed in bloods of a normal person and a breast cancer patient.

FIG. 29 illustrates a comparison of an expression rate for each CTC marker by targeting a stage 0 breast cancer patient.

FIG. 30 illustrates a comparison of an expression level for each CTC marker (Snail, E-cadherin, Cyclin D1, PTEN, and NPTN).

FIG. 31 illustrates a comparison of an HER2 expression rate in blood with a cancer-related marker in blood using RT-qPCR.

FIG. 32 illustrates an analysis of a correlation between HER2 expression and Ki67/hTER expression rate in blood.

FIG. 33 illustrates an analysis of a correlation between histological grade and hTERT/Ki67.

FIG. 34 illustrates a comparison of an expression rate of HER2 in blood with expression of histological HER2.

FIGS. 35 and 36 illustrate verification of expression rate of an epithelial antigen for each stage of a breast cancer patient.

FIGS. 37 to 39 illustrate verification of expression rates of cancer-related markers in cells for each stage of breast cancer.

### BEST MODE

Hereinafter, the present invention will be described in more detail with reference to unlimited Examples. However, the following Examples are described for exemplifying the present invention and it is not understood that the scope of the present invention is limited to the following Examples.

### Example 1

### Tissue Test Method for Screening Test for Therapeutic Agent

1) Material

Formalin fixed paraffin embedded (FFPE) tissues of 199 patients were used at Severance Hospital in Shinchon from 2010 to 2011. Expression of histological HER2 of the patients was verified by performing immunohistochemistry (IHC) and fluorescence in situ hybridization (FISH). Further, in order to verify the expression of HER2, the expression of HER2 was verified by using SK-BR3, MCF7, and MDA-MB 231 as breast cancer cell lines.

2) Immunohistochemistry (IHC)

A paraffin block was thin-sectioned with a thickness of 4 μm to be attached to a slide and sufficiently dried, and then immunohistochemistry (IHC) was performed by using a BenchMark ST (Ventana medical system, USA) automatic immunostaining device. A primary antibody was used by diluting polyclonal rabbit anti-human c-erbB-2 oncoprotein (A0485, DakoCytomation, Glostrup, Denmark) with 1:1,000. After the slide was stained by the method, it was determined that the stained slide was divided into four grades, that is, 0, 1+, 2+, and 3+ according to the degree that a HER2 protein was stained in a cell membrane of a cancer cell. Among the four grades, 0 and 1+ were diagnosed as HER2 negative, 3+ was diagnosed as positive, and 2+ was diagnosed as positive or by performing FISH according to clinical information of the patient.

3) Fluorescence in situ Hybridization (FISH)

In a HER2 IHC method, by targeting patients showing 2+, after a tissue block fixed with paraffin was thin-sectioned with a thickness of 4 μm by using a microtome to be attached to a slide, a test was performed according to a manufacturer's instructions by using a HER2 DNA probe kit (Vysis Inc, Downers Grove, Ill., USA) commercialized through deparaffinization and hydration processes. The HER2 expression was determined as positive according to a gene expression degree when an amplification index was 2.2 or more.

4) Total RNA Isolation in Separated Tissue

RNA was extracted by using a MagNApure LC RNA Isolation Kit III (Roche) as automatic nucleic acid extraction equipment after performing the deparaffinization process by using two pieces obtained by thin-sectioning a FFPE tissue with a thickness of 10 μm.

In the case of a cell line, after the number of cells in each cell line was adjusted to 1×10⁶, Total RNA was isolated by using Trizol according to a protocol of a manufacturer. The isolated Total RNA was quantified by using a NanoQuant system (TECAN).

5) cDNA preparation from isolated Total RNA and performance of Real-time PCR

a. cDNA Synthesis

0.5 to 3 μg of the isolated total RNA, 0.25 μg of random primer (Invitrogen), 250 μM of dNTP (Intron), Tris-HCl (pH 8.3) 50 mM, KCl 75 mM, MgCl₂ 3 mM, DTT 8 mM, and MMLV reverse transcription polymerase 200 units (Invitrogen) were added and mixed with DEPC-treated DW to have 30 μl of a final volume, and then reacted with a synthesis reaction solution for 10 min at 25° C., for 50 min at 37° C., and for 15 min at 70° C. in a thermocycler (ABI) to synthesize cDNA.

b. Performance of RT-qPCR

A composition of a reactant of Real-time PCR was prepared by adding 25 mM TAPS (pH 9.3 at 25° C.), 50 mM KCl, 2 mM MgCl₂, 1 mM 2-mercaptoethanol, 200 μM each dNTP, and 1 unit Taq polymerase (TAKARA), adding 10 μmole of a forward primer and a reverse primer, respectively, adding 10 μmole of probe, and adding 2 μl of the synthesized cDNA to have a final volume of 20 Each primer and a base sequence of a probe was illustrated in Table.

PCR reaction was performed once for 5 min at a denaturing temperature of 94° C. by using ABI 7500Fast (Applied Biosystem), 10 cycles was first performed for 30 sec at a denaturing temperature of 95° C. and an annealing temperature of 60° C. and then a cycle which was 30 sec at 95° C. and 40 sec at 55° C. was repetitively preformed 40 times. Further, a process of measuring fluorescence was added after each annealing process to measure an increased fluorescence value for each cycle.

6) Analysis of Result

A result of each test was analyzed by using 7500 software v2.0.4 (Applied Biosystem). SK-BR3 as breast cancer cells was diluted from 10⁵ to 1 cell in stages to draw a relative quantitation curve, and then expression levels were compared and quantified by using a Ct value to examine an expression rate. In this case, expression levels of each HER2 were compared based on an expression level of GAPDH, and an expression level of HER2 in each specimen and each cell line was indicated after determining a HER2 expression level of MDA-MB-231 as a HER2 negative breast cancer cell line as a reference of 1.

7) Verification of Amplification Through Software Analysis and Quantification of Amplified Product

An expression level of a specific gene of qRT-PCR was measured based on the following Relational Formula by using a comparative Ct method which was one of quantifying methods of the expression level and the Formula was embedded in ABI 7500 software-Bio-Rad CFX Manager Software and automatically calculated.

ΔΔCt=ΔCt(sample)−ΔCt(reference gene) [Relational Formula 1]

Herein, the Ct value represented a value of cycle in which amplification started to be distinctly increased during the PCR process.

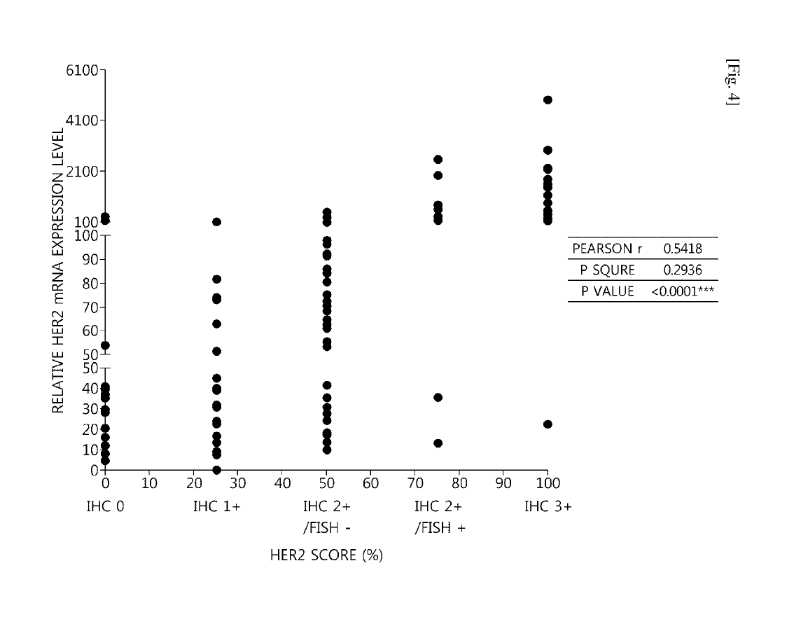

ΔΔCt means an mRNA expression ratio of a vertical axis in FIGS. 5 to 7.

Relational Formula of expression level analysis of HER2 in positive control group

ΔCt value of SKBR3=Ct value of HER2 in SKBR3−Ct value of reference gene(GAPDH) in SKBR3

ΔCt value of THP-1=Ct value of HER2 in THP-1−Ct value of reference gene (GAPDH) in THP-1

R(expression level)=ΔCt value of SKBR3-ΔCt value of THP-1 [Relational Formula 2]

Relational Formula of expression level analysis of HER2 for tissue sample of breast cancer patient

ΔCt value in breast cancer patient tissue=Ct value of HER2 in breast cancer patient tissue−Ct value of reference(GAPDH)gene in tissue

ΔCt value of THP-1=Ct value of HER2 in THP-1−Ct value of reference gene (GAPDH) in THP-1

R(expression level)=ΔCt value in breast cancer patient tissue−ΔCt value in THP-1 [Relational Formula 3]

The Ct value of the reference gene used in the test represented the Ct value for GAPDH and the reference gene may include another housekeeping gene in addition to GAPDH used in this test.

SKBR3: was a positive control and verified whether HER2 was actually overexpressed.

### Example 2

### Analysis of Cancer-Expression Marker in Blood

1) Specimens

Blood of 188 breast cancer patient provided from Severance Hospital of Yonsei University from 2011 to 2012 and blood of 50 normal donors without breast cancer were used.

2) Isolation of Cells from Blood of Patients

2 tube of blood were extracted from veins of a cancer patient and a normal person by using a tube including an EDTA anticoagulant. In order to prevent contamination from epithelial cells, first extracted 5 ml was discarded and next extracted 10 ml was used in the test. In order to prevent an mRNA damage from patient's blood, a lysis process of red blood cells as a first test process started within 4 hours after extracting the blood, a red blood cell lysis solution including NH₄Cl 154 mM, KHCO₃ 9 mM, and EDTA 0.1 mM was put with a volume of 5 times so as to lyse the red blood cells from the blood and left for 10 min at room temperature after vortex, and then centrifuging was performed for 10 min at 4° C. and 600 g and a supernatant was carefully discarded. In order to remove the remaining red blood cells, 10 ml of a RBC lysis buffer was added and left in ice for 5 min, and then the centrifuging was performed again for 2 minutes at 4° C. and 3000 rpm and the supernatant was carefully discarded, 1 ml PBS was added and a pellet was re-suspended and treated with RNase A (100 μg/ml) for 5 min in order to remove free nucleic acid in the blood.

3) Total RNA Isolation from Isolated Cells

The re-suspended pellet was centrifuged again for 2 min at 4° C. and 3000 rpm, the supernatant was discarded by pipetting, and then a Trizol reagent (Invitrogen) 1 ml was added to isolate the Total RNA according to a protocol of a manufacturer.

4) cDNA preparation from isolated Total RNA and performance of Real-time PCR

a. cDNA Synthesis

2 μg of the isolated total RNA, 0.25 μg of random hexamer (Invitrogen), 250 μM of dNTP (Cosmo gene tech), Tris-HCl (pH 8.3) 50 min, KCl 75 min, MgCl₂ 3 min, DTT 8 min, and MMLV reverse transcription polymerase 200 units (Invitrogen) were added and mixed with DEPC-treated DW to have 20 μl of a final volume, and then reacted with a synthesis reaction solution for 10 min at 25° C., for 50 min at 37° C., and for 15 min at 70° C. in a thermocycler (ABI) to synthesize cDNA.

b. Performance of Real-Time PCR

A composition of a reactant of Real-time PCR was prepared by adding 25 min TAPS (pH 9.3 at 25° C.), 50 min KCl, 2 min MgCl₂, 1 min 2-mercaptoethanol, 200 μM each dNTP, and 1 unit Taq polymerase (TAKARA), adding 10 μmole of a forward primer and a reverse primer, respectively, adding 10 μmole of a probe, and adding 2 μl of the synthesized cDNA to have a final volume of 20 μl. Each primer and a base sequence of a probe were as follows.

TABLE 1

| SEQ ID NO: | HER605-F | AACCTGGAACTCACCTACCTGCCCAC | |
|---|---|---|---|
| 1 | | | |
| SEQ ID NO: | HER689-R | CGATGAGCACGTAGCCCTGCAC | |
| 2 | | | |
| SEQ ID NO: | HER612-F | AACTCACCTACCTGCCCACCAAT | |
| 3 | | | |
| SEQ ID NO: | HER633-R | CACGTAGCCCTGCACCTCCT | |
| 4 | | | |
| SEQ ID NO: | HER637-P | CAG CCT GTC CTT CCT GCA GGA | FAM- |
| 5 | | TAT C | BHQ1 |
| SEQ ID NO: | HER2-F1 | AAG CAT ACG TGA TGG CTG GTG T | |
| 6 | | | |
| SEQ ID NO: | HER2-R1 | TCT AAG AGG CAG CCA TAG GGC | |
| 7 | | ATA | |
| SEQ ID NO: | HER2-P1 | ATA TGT CTC CCG CCT TCT GGG | FAM- |
| 8 | | CAT CT | BHQ1 |
| SEQ ID NO: | HER2-P2 | CAT CCA CGG TGC AGC TGG TGA | FAM- |
| 9 | | CAC A | BHQ1 |
| SEQ ID NO: | ER(6118)-F1 | TCAAATGCCAAATTGTGTTTGATGGA | |
| 10 | | | |
| SEQ ID NO: | ER(6201)-R2 | GCTGCGACAAAACCGAGTCACATCA | |
| 11 | | | |
| SEQ ID NO: | ER(6145)-P | TAATATGCCCTTTTGCCGATGCATAC | FAM- |
| 12 | | T | BHQ1 |
| SEQ ID NO: | ER(6122)-F | ATGCCAAATTGTGTTTGATGGAT | |
| 13 | | | |
| SEQ ID NO: | ER(6197)-R | CGACAAAACCGAGTCACATCAGTAA | |
| 14 | | T | |
| SEQ ID NO: | P | at gcccttttgc cgatgca | FAM- |
| 15 | | | BHQ1 |
| SEQ ID NO: | PR(1925)-F | AGTCAGAGTTGTGAGAGCACTGGA | |
| 16 | | | |
| SEQ ID NO: | PR(2006)-R | CTGGCTTAGGGCTTGGCTTTCATT | |
| 17 | | | |
| SEQ ID NO: | PR(1950)-P | TGCTGTTGCTCTCCCACAGCCAGT | FAM- |
| 18 | | | BHQ1 |
| SEQ ID NO: | PR(1932)-F | GTTGTGAGAGCACTGGATGCTGTT | |
| 19 | | | |
| SEQ ID NO: | PR(2010)-R | AATCTCTGGCTTAGGGCTTGGCTT | |
| 20 | | | |
| SEQ ID NO: | PR(1964)-P | ACAGCCAGTGGGCGTTCCAAATGA | FAM- |
| 21 | | | BHQ1 |
| SEQ ID NO: | GAPDH-F | cca tct tcc agg agc gag atc c | |
| 22 | | | |
| SEQ ID NO: | GAPDH-R | atg gtg gtg aag acg cca gtg | |
| 23 | | | |
| SEQ ID NO: | GAPDH-P | tcc acg acg tac tca gcg cca gca | Cy5- |
| 24 | | | BHQ2 |
| SEQ ID NO: | EPCAM(158)- | GCCAGTGTACTTCAGTTGGTGCAC | |
| 25 | F | | |
| SEQ ID NO: | 240-R | CATTTCTGCCTTCATCACCAAACA | |
| 26 | | | |
| SEQ ID NO: | 186-P | TACTGTCATTTGCTCAAAGCTGGCTG | FAM- |
| 27 | | CCA | BHQ1 |
| SEQ ID NO: | CK19-F | GATGAGCAGGTCCGAGGTTA | |
| 28 | | | |
| SEQ ID NO: | CK19-R | TCTTCCAAGGCAGCTTTCAT | |
| 29 | | | |
| SEQ ID NO: | CK19-P | CTGCGGCGCACCCTTCAGGGTCT | FAM- |
| 30 | | | BHQ1 |
| SEQ ID NO: | hTERT-F | TGACGTCCAGACTCCGCTTCAT | |
| 31 | | | |
| SEQ ID NO: | hTERT-R | ACGTTCTGGCTCCCACGACGTA | |
| 32 | | | |
| SEQ ID NO: | hTERT-P | GCTGCGGCCGATTGTGAACATGGA | FAM- |
| 33 | | | BHQ1 |
| SEQ ID NO: | Ki67-F | TAATGAGAGTGAGGGAATACCTTTG | |
| 34 | | | |
| SEQ ID NO: | Ki67-R | AGGCAAGTTTTCATCAAATAGTTCA | |
| 35 | | | |
| SEQ ID NO: | Ki67-P | GGCGTGTGTCCTTTGGTGGGCA | FAM- |
| 36 | | | BHQ1 |
| SEQ ID NO: | Vimentin-F | ATGTTGACAATGCGTCTCTGGCA | |
| 37 | | | |
| SEQ ID NO: | Vimentin-R | ATT | |
| 38 | | TCCTCTTCGTGGAGTTTCTTCAAA | |
| SEQ ID NO: | Vimentin-P | TGACCTTGAACGCAAAGTGGAATCTT | FAM- |
| 39 | | TGC | BHQ1 |
| SEQ ID NO: | CyclinD1-F | TGAACAAGCTCAAGTGGAACCTGG | |
| 40 | | | |
| SEQ ID NO: | CyclinD1-R | CTGTTTGTTCTCCTCCGCCTCTGG | |
| 41 | | | |
| SEQ ID NO: | CyclinD1-P | CCCGCACGATTTCATTGAACACTTCC | FAM- |
| 42 | | | BHQ1 |
| SEQ ID NO: | E-cad422F | GGGCACAGATGGTGTGATTACAGTC | |
| 43 | | | |
| SEQ ID NO: | E-cad500R | CCCAGGCGTAGACCAAGAAATG | |
| 44 | | | |
| SEQ ID NO: | E-cad452P | GGCCTCTACGGTTTCATAACCCACAG | FAM- |
| 45 | | ATC | BHQ1 |
| SEQ ID NO: | Snail6501-F | TGTGACAAGGAATATGTGAGCCTGG | |
| 46 | | | |
| SEQ ID NO: | Snail6582-R | CGCAGATCTTGCAAACACAAGG | |
| 47 | | | |
| SEQ ID NO: | Snail6530-P | CCTGAAGATGCATATTCGGACCCACA | FAM- |
| 48 | | CATT | BHQ1 |
| SEQ ID NO: | PTEN1828F | GTCTGAGTCGCCTGTCACCATTT | |
| 49 | | | |
| SEQ ID NO: | PTEN1908R | CGCCGTGTTGGAGGCAGTAG | |
| 50 | | | |
| SEQ ID NO: | PTEN1858P | TGGGAACGCCGGAGAGTTGGTCTCT | FAM- |
| 51 | | | BHQ1 |
| SEQ ID NO: | NPTN651F | ACCAGTGAAGAGGTCATTATTCGAGA | |
| 52 | | CA | |
| SEQ ID NO: | NPTN739R | TATGTAAGGGTGTGAGAGCTGGAGGT | |
| 53 | | | |
| SEQ ID NO: | NPTN681P | CCTGTTCTCCCTGTCACCCTGCAGTGT | FAM- |
| 54 | | AAC | BHQ1 |

Table 1 illustrated primers and base sequences of probes.

PCR reaction was performed once for 5 min at a denaturing temperature of 94° C. by using ABI 7500Fast (Applied Biosystem) and a cycle of 30 sec at a denaturing temperature of 95° C. and 20 sec at an annealing temperature of 55° C. was repetitively performed 40 times. Further, a process of measuring fluorescence was added after each annealing process to measure an increased fluorescence value for each cycle.

5) Analysis of Result

A result of each test was analyzed by using 7500 software v2.0.4 (Applied Biosystem). In the case of EpCAM and CK19 as cell surface antigens, positive and negative were divided based of a Ct value of RT-qPCR. When the Ct values of two markers were 38 or less, it was determined as positive, and when the Ct values were 38 or more, it was determined as negative. In the case of each marker, expression levels in a patient group were compared and quantified based on expression levels of HER2, hTERT, and Ki67 expressed in the blood of the normal person to examine the expression rate. In this case, the expression levels of markers were compared based on the expression level of GAPDH.

The result of the Example is as follows.

1. Use of RT-qPCR in Tissue Test for Screening Therapeutic Agent

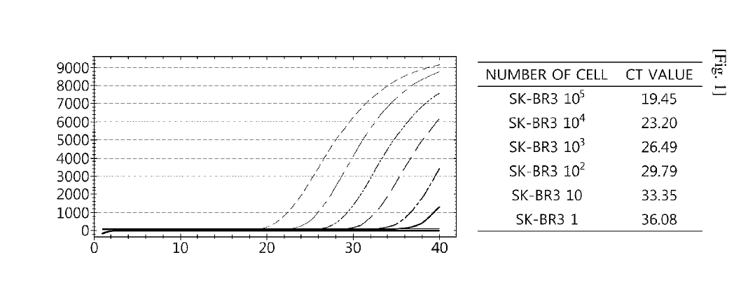

1) Comparison of HER2 expression levels for each cell line using RT-qPCR Expression sensitivity of HER2 was verified by using SK-BR-3 as a HER2 positive breast cancer cell line. As a result, as illustrated in FIGS. 1 and 2, the sensitivity using RT-qPCR of HER2 may verify sensitivity capable of detecting SK-BR-3 cells up to one.

For higher sensitivity, the method was compared with an existing method by using a one-tube nested PCR condition of HER2 and as a result, the SK-BR-3 cells were detected up to one, but it was verified that the Ct value resulted from each cell line was highly represented by about 10⁴. Accordingly, in the present invention, a clinical test was performed by using the one-tube nested PCR condition.

Further, expression levels of HER2 in respective cell lines were compared by using SK-BR3, MCF7, and MDA-MB-231 cell lines as breast cancer cell lines. When the HER2 expression of the MDA-MB-231 cell line as the HER2 negative cell line was set as 1, it was verified that the HER2 expression level of MCF7 was shown as approximately 5.4 and the HER2 expression level of SK-BR-3 was shown as approximately 56.9.

2) Setting of Clinical Cut-Off

After HER2 RT-qPCR was performed by using FFPE specimens of 199 breast cancer patients provided from Severance Hospital in Shinchon, the result was compared with the IHC Score and the FISH result of the breast cancer patients. Scores were designated to 0 in the case of IHC 0, 25 in the case of IHC 1+, 50 in the case of IHC 2+ and FISH negative, 75 in the case of IHC 2+ and FISH positive, and 100 in the case of IHC 3+, respectively, and then a correlation analysis between the result and the result of HER2 RT-qPCR was performed.

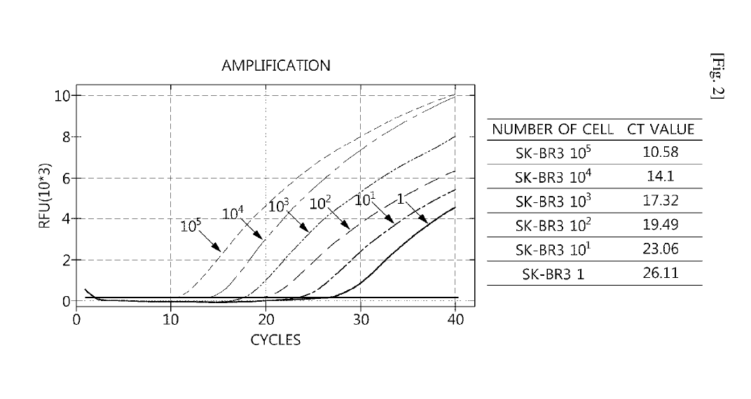

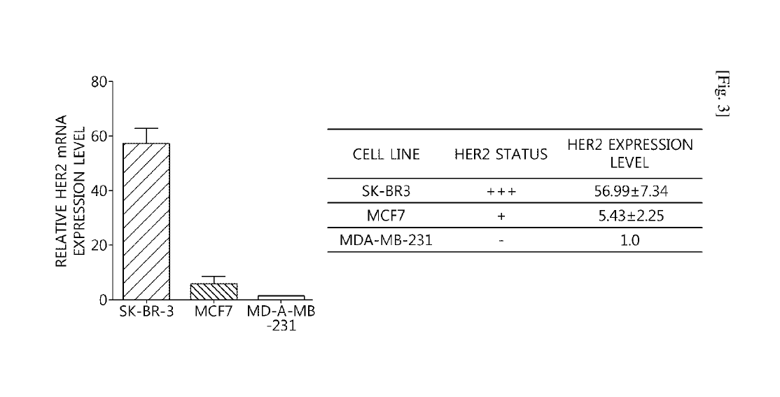

In FIG. 3, it could be seen that a Pearson r value was shown as 0.5418 R square and 0.2936 p value was shown as <0.0001 to have a correlation between RT-qPCR and IHC results.

Further, in the test using the FFPE specimens, RNA quality of the specimens was very important. The specimens having high RNA quality may show the accurate result, while if the RNA quality is low, the result of false-positive or false-negative may be shown. Accordingly, in the present invention, the degree of the RNA quality was indicated based on the expression level of GAPDH. As illustrated in FIGS. 5 to 7, it was verified that when the expression degree of GAPDH was classified based on the Ct value of RT-qPCR, as the Ct value of GAPDH have a low value, a more accurate result was shown.

In this case, a receiver operating characteristic (ROC) curve is a graph showing performance of a determined result (binary classifier) of any test, and has a true positive rate (TPR) or sensitivity as a y axis and a false positive rate (FPR) or 1-specificity as an x axis.

That is, the result is calculated as TRP=y axis=sensitivity=(TP/(TP+FN) and FPR=x axis=1-specificity=1−[TN/(TN+FP)].

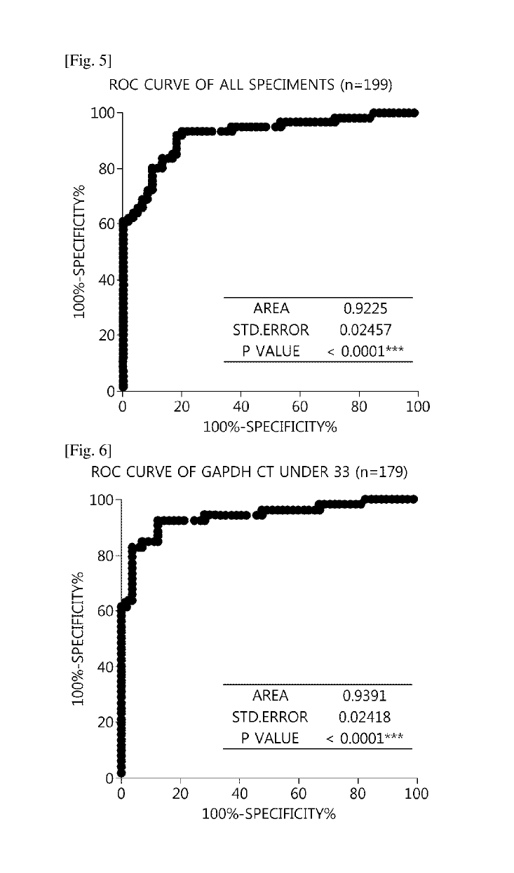

In FIGS. 5 to 7, in all of the specimens, it was seen that in specificity and sensitivity analysis, a case of analyzing specimens having the Ct value of 30 or less of GAPDH (FIG. 7) was higher than a case of analyzing only the specimens having the Ct value of 33 or less of GAPDH (FIG. 6) and a case of analyzing all of the specimens regardless of the result of GAPDH (FIG. 5). Based on the result, in the present invention, the GAPDH Ct value has only the specimens having the GAPDH Ct value of 30 or less and a clinical Cut-off analysis was performed, and the result was illustrated in FIG. 5.

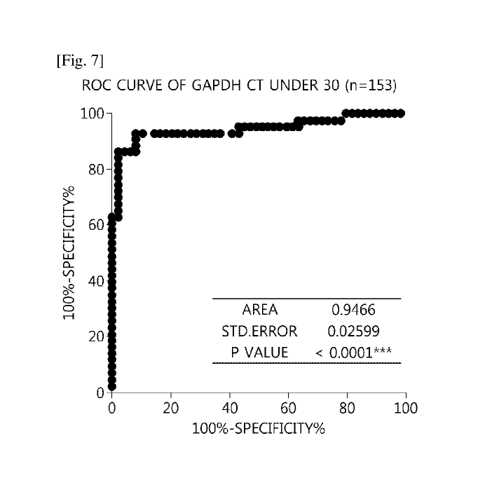

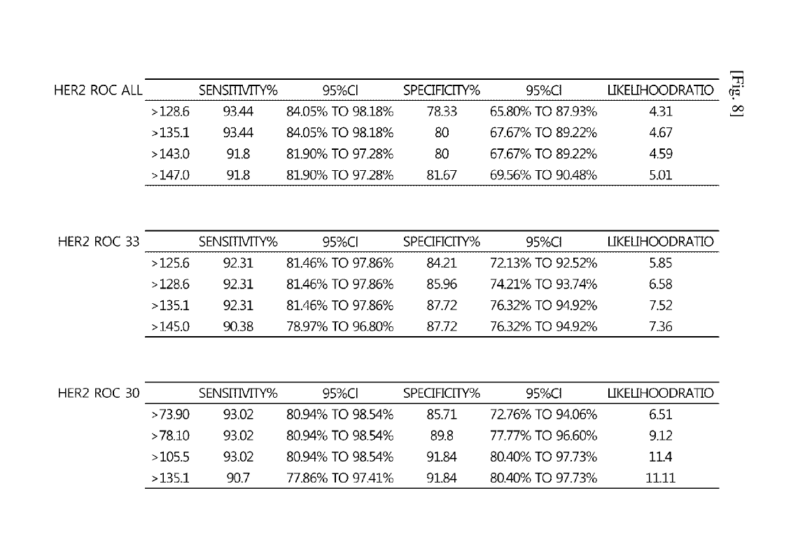

In FIG. 8, it was seen that when the GAPDH Ct value was 30 or less, the sensitivity and the specificity were highest (sensitivity of 93.02 and specificity of 91.84), and the Cut-off thereof was 105.5. A result of analyzing the clinical specimens using the designated Cut-off may be illustrated in FIG. 9.

As illustrated in FIG. 9, in IHC 0/1+, the result was calculated by analyzing IHC 2+/FISH positive specimens as negative and IHC 3+ specimens as positive. The IHC 2+/FISH negative specimens were yet clinically discussed and excluded from calculation of the exception result. According to N Engl J Med. 2008 Mar. 27; 358(13):1409-11, when Herceptin as a HER2 target therapeutic agent was administrated to the IHC 2+/FISH negative patient, it was verified that there was an effect, and thus the corresponding patients may not be defined as HER2 positive or negative. Accordingly, it could be seen that the sensitivity and the specificity of the present invention except for the corresponding part were 93.0% and 91.8%, respectively.

3) RT-qPCR Method for Hormone Receptor Test

Even in addition to a size and lymph-node metastasis of cancer known as a prognosis predictor of breast cancer, and histologic differentiation of the cancer, positive of a hormone receptor (ER; estrogen receptor, PR; progesterone receptor) plays a very important role in determination of prognosis and treatment. A treatment of an anti-hormone such as tamoxifen after operating a hormone receptor-positive patient was an important adjuvant therapy to enhance a survival rate, but a lot of treated patients were recurred and it means resistance to anti-hormone treatment. A loss of the hormone receptor positive is known as one of important resistant expression mechanisms and this suggests a change in hormone receptor according to the anti-hormone treatment. In the breast cancer treatment process, even with respect to both primary breast cancer after operating and recurrent breast cancer, an immunohistochemistry test for expression of the hormone reception has been basically performed. Presence of the hormone receptor is known as a good prognosis factor and it is known that triple negative breast cancer has bad prognosis. By a change from positive to negative of the hormone receptor and a change in which the triple negative was high, in recurrent cancer compared with primary cancer, there is no chance to receive appropriate anti-hormone treatment and target treatment such as Herceptin and there is a relation with bad prognosis. Accordingly, the hormone receptor verified the sensitivity through RT-qPCR.

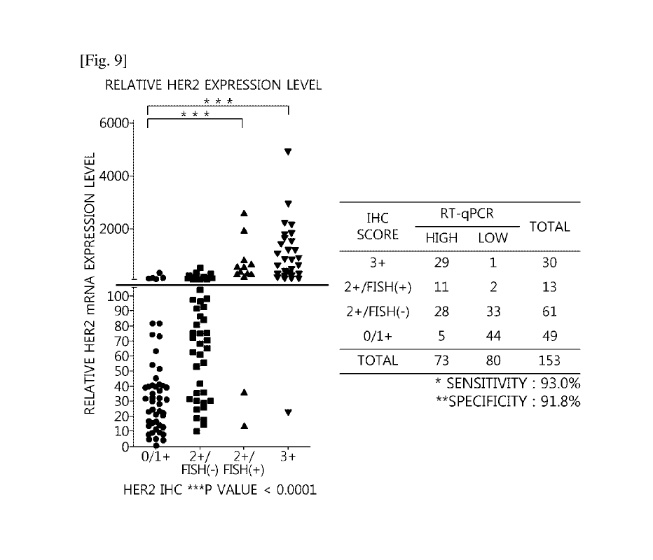

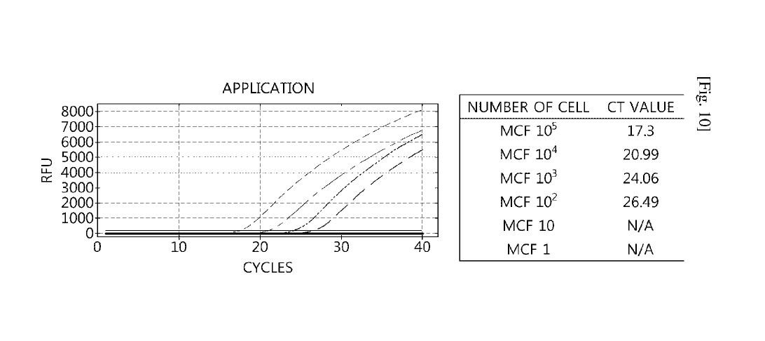

In FIG. 10, the sensitivity of an estrogen receptor (ER) was verified by using the one-tube nested PCR condition in the MCF7 cell line and detected in 10² cells. In FIG. 11, the sensitivity of a progesterone receptor (PR) was verified by using the one-tube nested PCR condition in the MCF7 cell line and detected in 10² cells, but the Ct value as the result value was highly shown. FIG. 12 illustrates sensitivity of the hormone receptors of both the ER and PR and it could be seen that the sensitivity was detected in 10¹ cells and the sensitivity was highly shown as compared with the case of performing an individual test like FIG. 10 or 11.

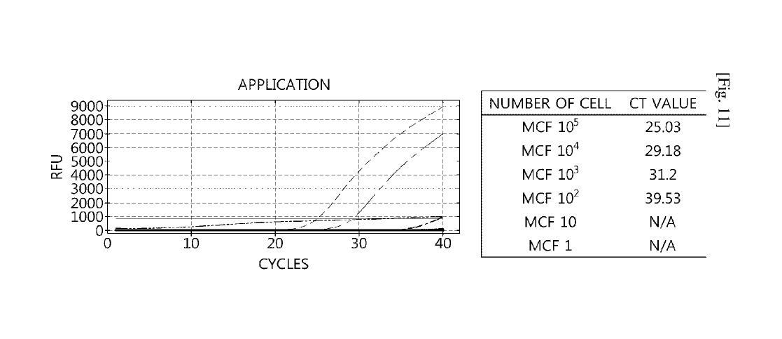

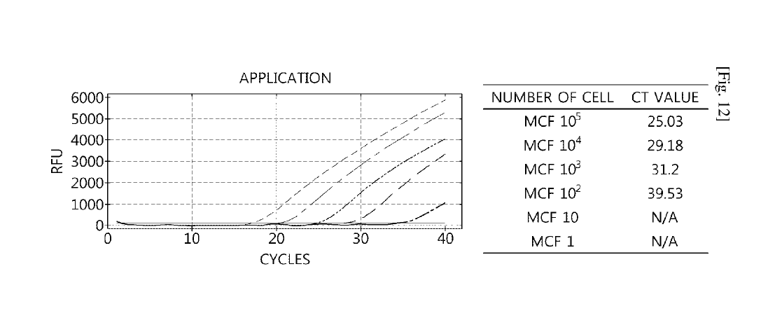

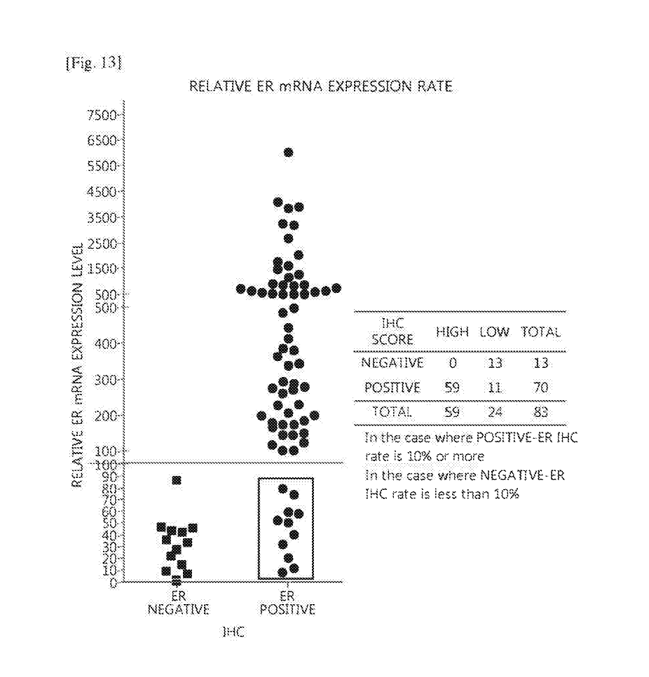

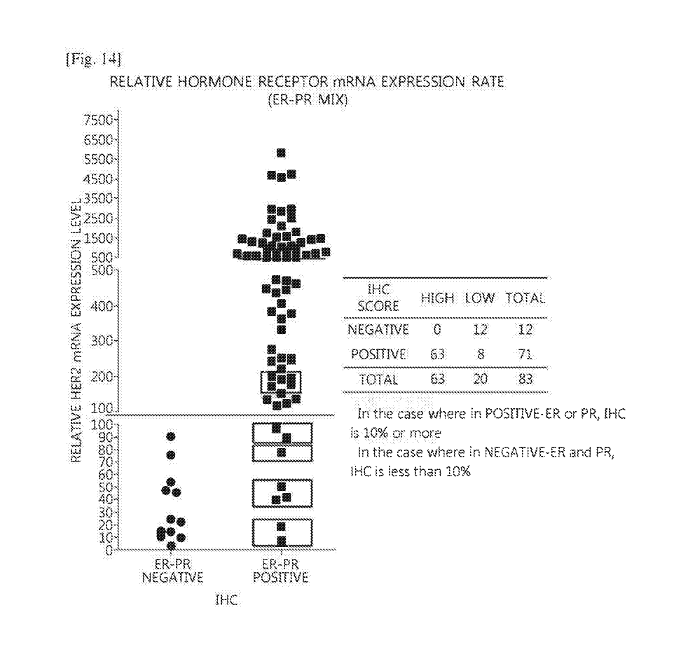

Further, as a result of comparing clinical tissue samples, in the case of performing only the ER, it can be seen that ER IHC positive of 10% or more was shown in 59 specimens (71.1%) (see FIG. 13), but in an ER-PR mixed hormone receptor test, the ER IHC positive was shown in 63 specimens (75.9%) and 4 specimens (4.8%) were positive (see FIG. 14).

2. Analysis of HER2 Expression and Cancer-Expression Marker in Blood

1) Comparison of Sensitivity and Specificity

In order to determine whether to detect the expression of HER2 in blood, in the present invention, after SK-BR-3 as the breast cancer cell line overexpressing HER2 was mixed with the blood of the normal person without breast cancer, a diagnosis of cancer cells in the blood was verified through RNA extraction.

As illustrated in FIG. 15, it could be seen that the sensitivity of HER2 was high until the cell of SK-BR-3 mixed with the blood was one.

Further, in order to verify the expression of HER2 in the blood, the expression of HER2 was verified by using 50 normal persons and 188 breast cancer patients provided from the Shinchon Severance Hospital. As a result, it could be seen that in the normal persons, the expression was shown low as about 0 to 1.5 and in the breast cancer patients, various expressions were shown from 0 to 355. Among them, patients having the HER2 expression of 10 or more in the blood were divided into positive and patients having the HER2 expression of 10 or less were divided into negative.

As illustrated in FIG. 16, it was verified that in the normal persons, the overexpression of HER2 was not shown, while in 39 persons (20.7%) of 188 breast cancer patients, the overexpression of HER2 was shown.

2) Comparison of Expression Levels of Other Cancer Markers in Blood

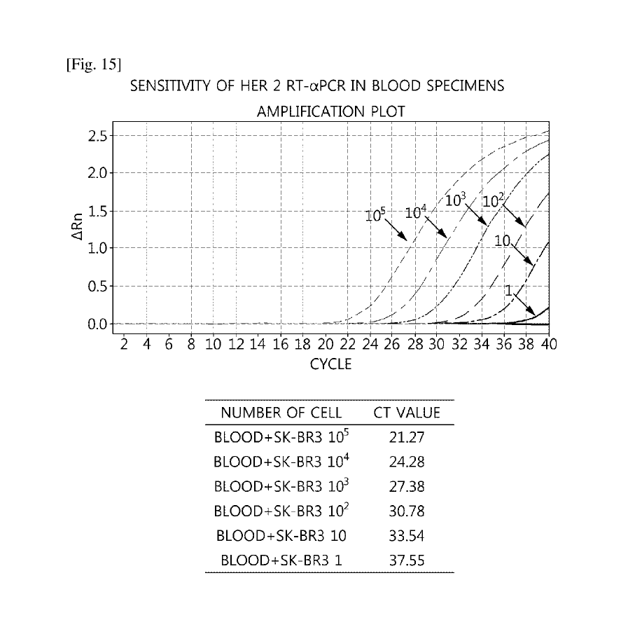

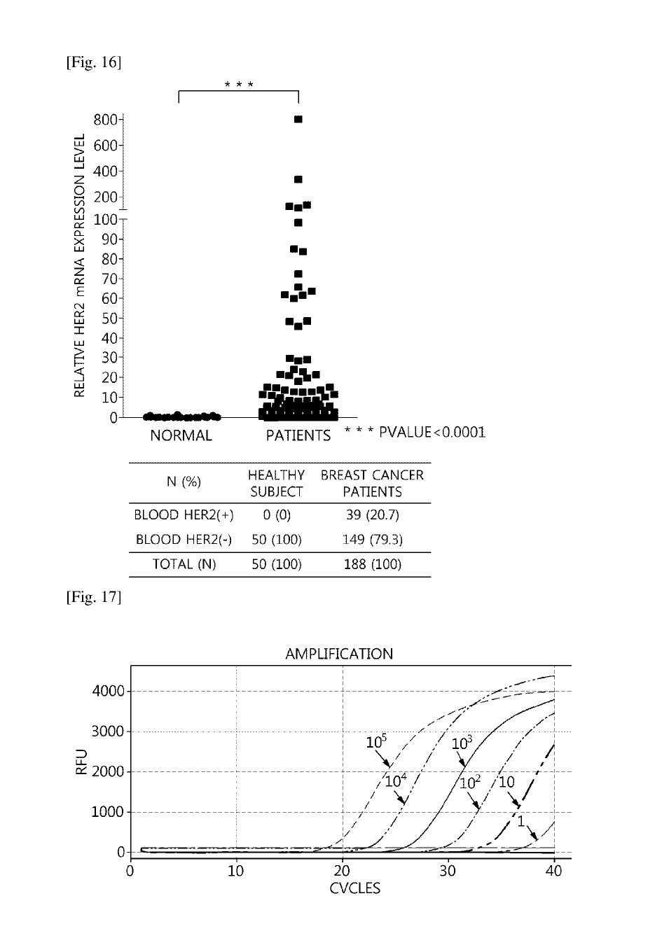

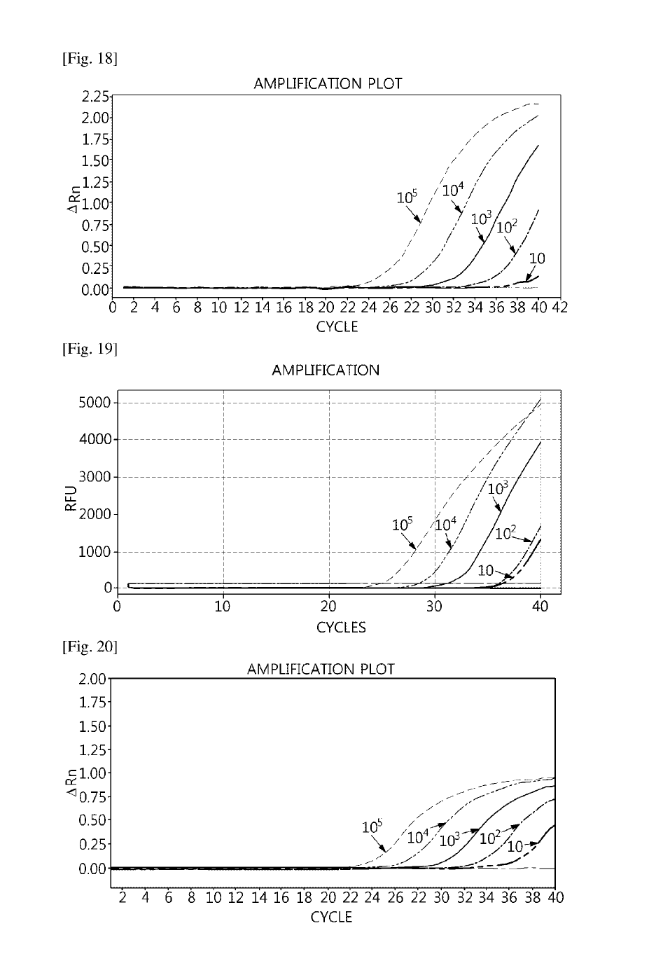

In order to verify whether the expression of HER2 was actually expressed by cancer cells in the blood, expression of other cancer cell-related markers was verified. Whether cancer cells were present in the blood was verified by using EpCAM (FIG. 17) and Cytokeratin 19 (FIG. 18) which were epithelial antigen markers and hTERT (FIG. 19) and Ki67 (FIG. 20) which were intracellular cancer-related markers. The sensitivity of EpCAM and CK19 was verified by using SK-BR-3 and the sensitivity of hTERT was verified by using MDA-MB-231. Finally, the sensitivity of Ki67 was verified by using MCF7.

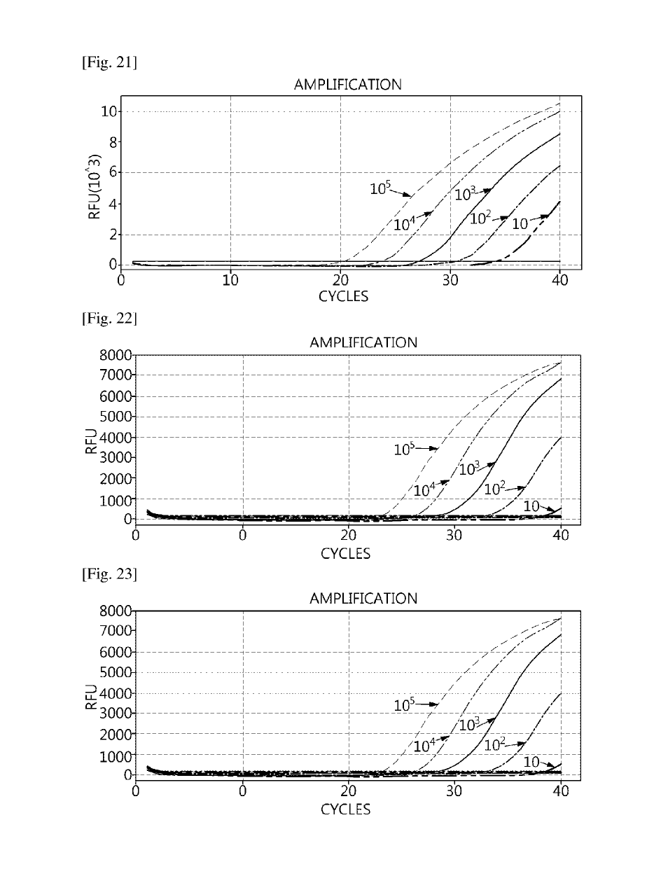

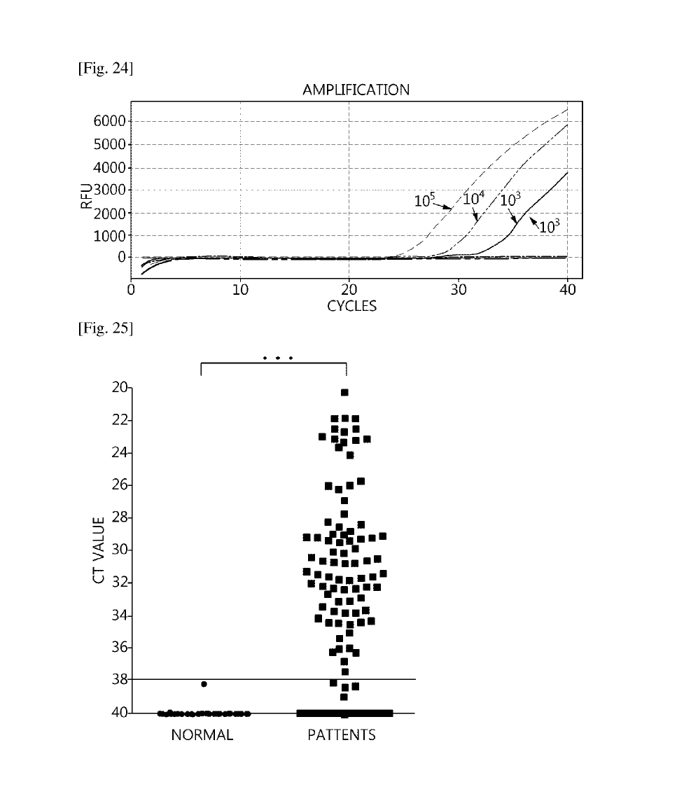

Vimentin as an intermediate filament protein related with mesenchymal-origin cells such as fibroblast cells or hematopoietic cells was not present in most of normal epithelial cells and the vimentin expression in a cytoplasm is widely distributed and a clear perinuclear & subplasmalemmal accentuation was frequently shown. The presence of the vimentin has a property of epithelial cells which may be independently survived. Accordingly, determining whether the vimentin and the cytokeratins were expressed may be used as an important marker capable of determining aggressiveness and metastatic ability of the breast cancer to verify the sensitivity by using MDA-MB-231 in order to verify the expression of the marker (see FIG. 21). Cyclin D1 is an important cell cycle control protein factor in development and performance, there is a correlation between an expression frequency of the Cyclin D1 and a clinicopathological prognostic factor of tumor, and overexpression of the Cyclin D1 is associated with positive expression of the estrogen receptor. Accordingly, the sensitivity was verified by using SK-BR3 in order to verify the expression of the marker (see FIG. 22). Phosphatase and tensin homolog deleted on chromosome ten (PTEN) as a tumor suppressor gene called a mutated in multiple advanced cancers (MMAC-1a) or TGF-β regulated and epithelial cell enriched phosphatase (TEP-1) is known to play an important role in maturation of embryo, movement of cells, and cell apoptosis in addition to suppressing a growth of cancer cells, and acts as lipid phosphatase adjusting a signaling pathway of an epidermal growth factor receptor. That is, the PTEN serves to adjust a phosphatidylinositol 3-kinase (PI3-kinase)/Akt pathway related with growth and survival of cells to suppress the progress of the cell division cycle and suppress the growth of the cell by causing G1 stop. Further, the PTEN dephosphorylates focal adhesion kinase (FAK) to cause movement and distribution of cells and focal adhesion. Since it was reported that the loss of the PTEN has a correlation between tumor progression and metastasis, whether the marker was expressed was verified (see FIG. 23). Neuroplastin is a protein encoded by a human NPTN gene. The Neuroplastin known as NPTN, GP55, GP65, stromal cell-derived receptor (SDR1), and np55 or SDFR1 as a type1 transmembrane protein belonging to an Ig superfamily interacts between cells or interacts in a cell substrate. In breast cancer, overexpression of the neuroplastin increases the growth of the cancer and angiogenesis to play an important role in cancer metastasis and thus the expression was verified by using the corresponding marker (see FIG. 24).

As described above, in the EpCAM, the sensitivity was verified up to one cell in the blood, in the CK19, the hTERT, the Ki67, the Vimentin, and the Cyclin D1, the sensitivity was verified up to 10 cells in the blood, and in the PTEN and the NPTN, the sensitivity was verified in 10² cells in the blood.

Further, as illustrated in FIG. 25, in the EpCAM and the CK19, the expression was compared by using the Ct value in the normal persons and the breast cancer patients and in the hTERT and the Ki67, a relative expression rate in the blood was compared in the normal persons and the patient group.

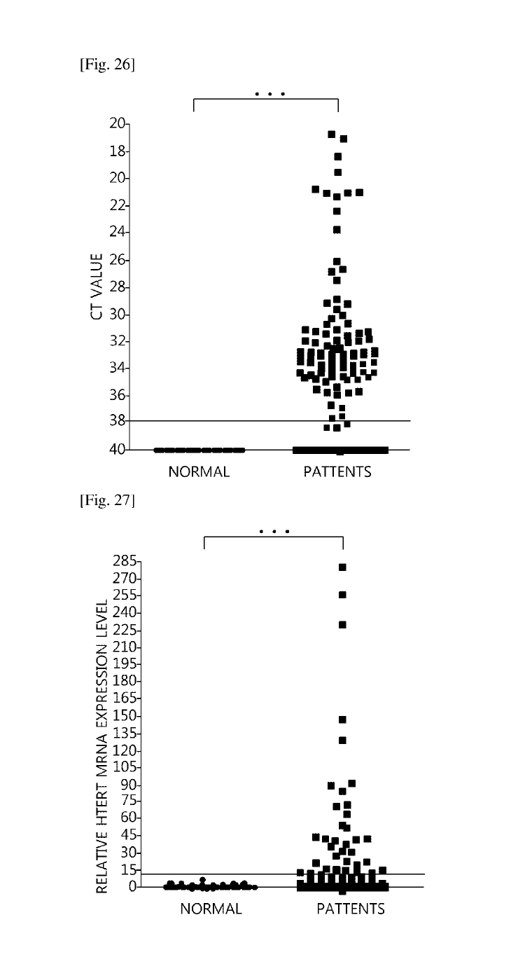

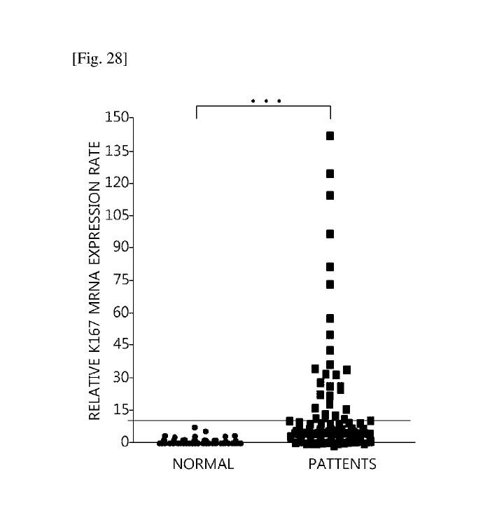

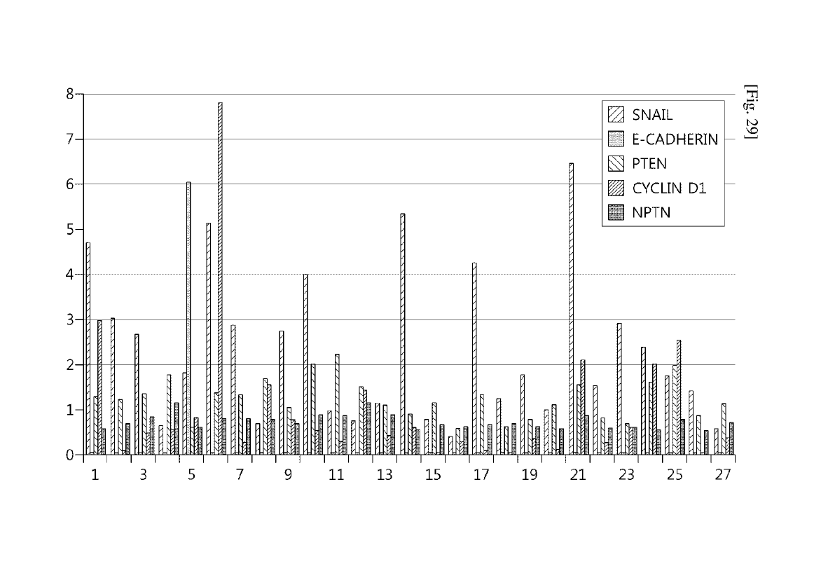

As illustrated in FIGS. 26 to 28, it was verified that in the EpCAM and the CK19, all of the normal persons except for one person have a low Ct value of 39 or more, while in the breast cancer patient group, patients having a high Ct value of 28 or less are present. In the EpCAM, it was verified that the positive rate of 45.2% was shown, and in the CK19, it was verified that the positive rate of 50.5% was shown. Further, when verifying the expression levels of the hTERT and the Ki67, it was verified that in the normal persons, there was no person having a relative expression level of 10 or more of both the hTERT and the Ki67, while in the breast cancer patient group, the hTERT had a high expression rate of 10 or more in 20.7% patients and the Ki67 had the high expression rate of 10 or more in 13.8% patients.

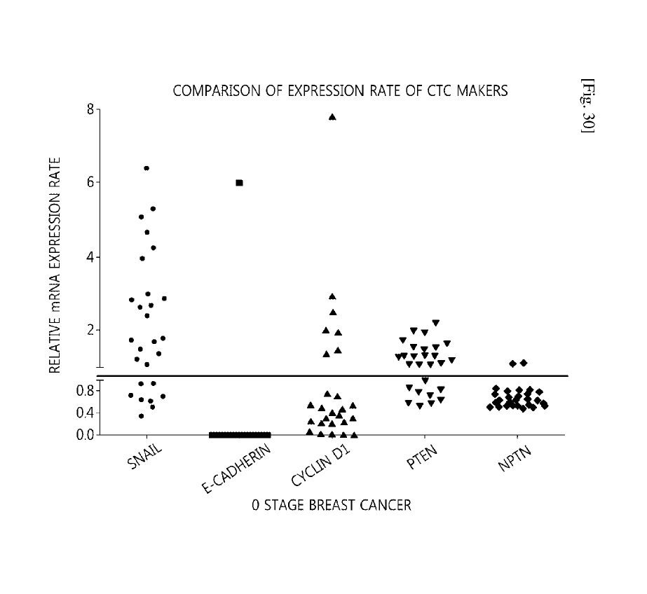

In the Cyclin D1, the amplification of the Cyclin D1 gene was reported in 15 to 20% of the breast tumor, and even in the test, when verifying the expression by targeting stage 0 breast cancer patients, the Cyclin D1 was highly expressed as 7/27 (25.9%). In the PTEN, when verifying the expression by targeting stage 0 breast cancer patients, the PTEN was expressed as 18/27 (66.7%), but the 9/27 (33.3%) was not expressed, and 9 patients need a follow-up test because the loss of the PTEN had a correlation between the tumor progression and the metastasis (see FIG. 29). Since an adhesion molecule plays an important role in invasion and metastasis of the cancer cells, an interest in the adhesion molecule is high in prediction of the progression process of the cancer.

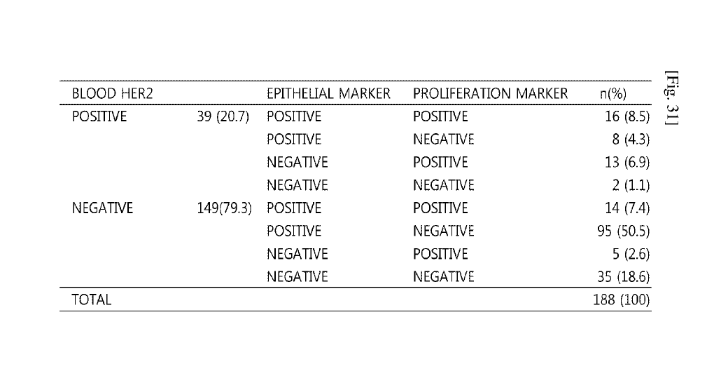

In the present invention, whether to express cadherin and Snail was verified by targeting stage 0 breast cancer patients. As a result, in the Snail, the positive expression was shown in 18/27 (66.7%), but in the E-cadherin, the positive reaction was shown only in 1/27 (3.7%). The Snail had a similar result to the report that the Snail was involved in a relatively early stage of a tumor forming process and was used as an initial diagnosis marker, and the E-cadherin had an expression of an inverse correlation with the Snail (see FIG. 30).

3) Comparison of expression rate of breast cancer-related marker with HER2 expression in blood

Which relationship between expression rates of specimens of patients expressing the HER2 in the blood and cancer-related markers used in the present invention was present was verified.

As illustrated in FIG. 31, it was verified that all of patients expressing the HER2 in the blood except for two patients were expressed simultaneously with an epithelial antigen or a cancer-related intracellular marker. It was verified that high expression of the HER2 in the blood was actually shown by the presence of cancer cells overexpressing the HER2.

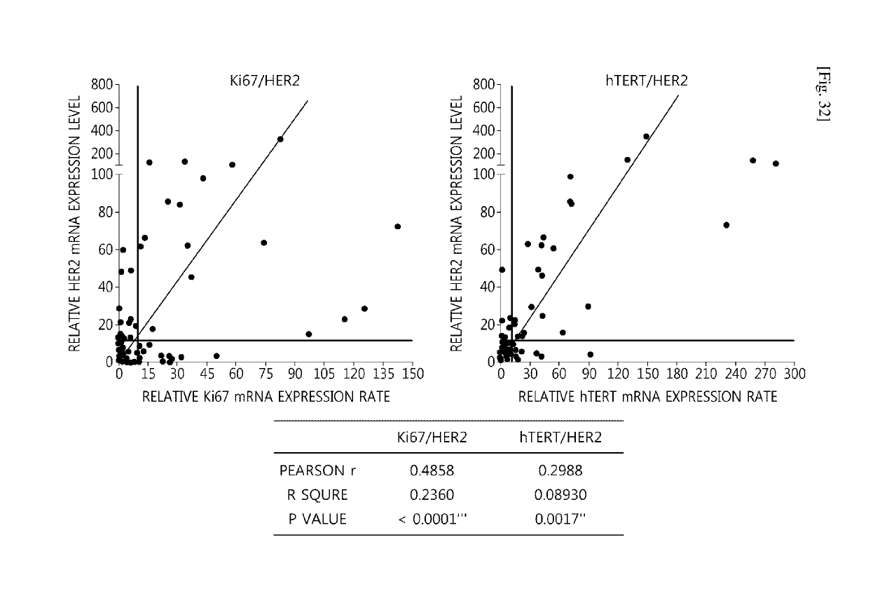

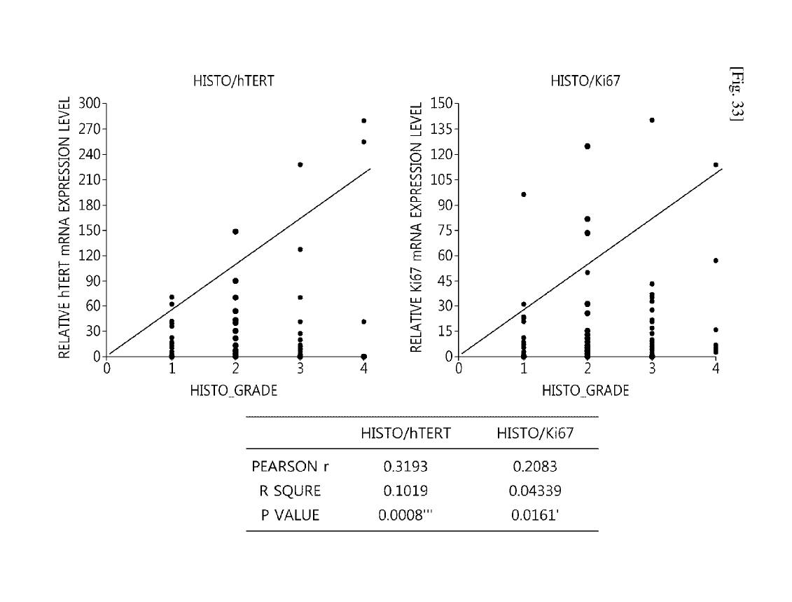

Particularly, it was verified that the expression of the HER2 in the blood had a low correlation with the expression of the Ki67 and the hTERT in the blood (see FIG. 32). As a result, it can be seen that overexpression of the HER2 had a correlation with malignance of cancer cells, and it was verified in FIG. 33 that as a histological grade of the cancer cells was gradually deteriorated, the expression of the Ki67 and the hTERT had a correlation to some degree.

4) Comparison of Histological Test HER2 Result with Expression of HER2 in Blood

Which the expression rates of HER2 expressed in the blood and HER2 using a histological test method have a relationship was compared by using the RT-qPCR.

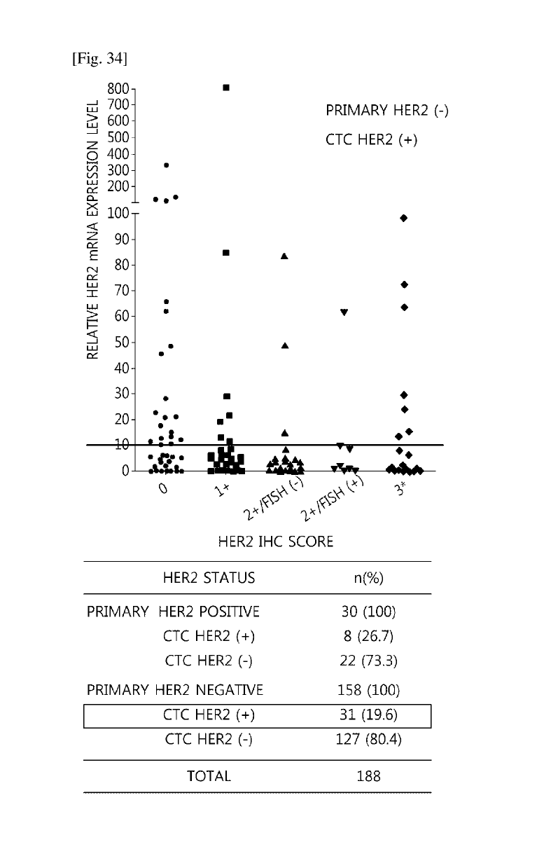

As illustrated in FIG. 34, it can be seen that the expression of the HER2 in the blood had a large difference from the histological HER2 test method. In the following Table in the drawing, a part illustrated by a red square represented patients having the HER2 negative in the histological test result, but patients having high HER2 expression in the blood. It was verified that the ratio was a ratio of 19.6% of the HER2 negative patients and high enough to occupy 1/5 of the entire ratio. It could be seen that the patients were associated with malignance of the cancer cells. Accordingly, it can be seen that the treatment is possible by administrating Herceptin as a target therapeutic agent of HER2 to patients having high expression of the HER2 in the blood.

5) Analysis of Cancer Marker in Blood for Each Breast Cancer Stage

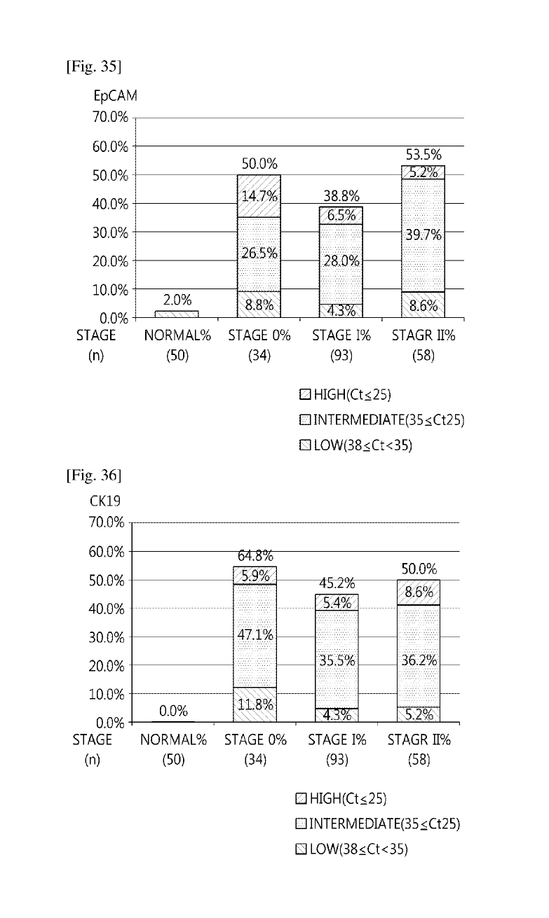

Analysis for breast cancer stage using cancer markers in the blood was performed. In FIGS. 35 and 36, it was verified that in the case of an epithelial antigen, there is no large difference for each stage of breast cancer. However, in stage 0, it was verified that a high expression rate was shown. It is considered that this may be associated with early detection of a breast cancer patient.

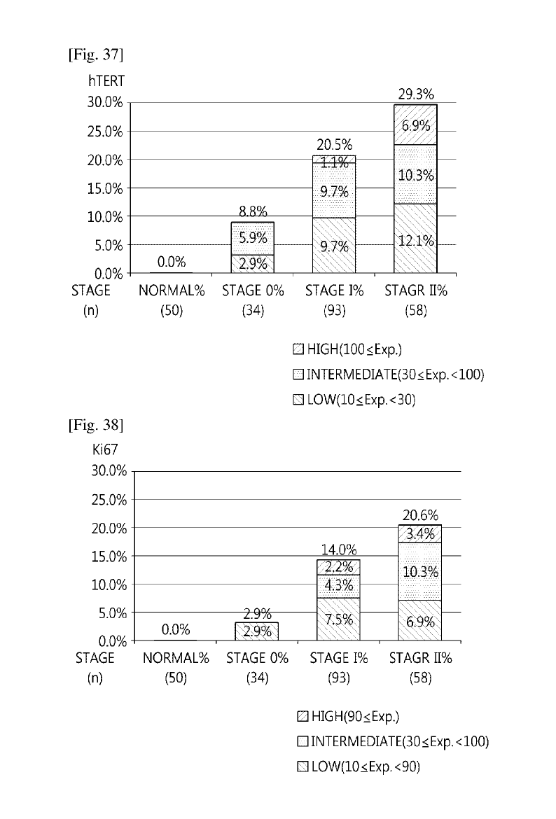

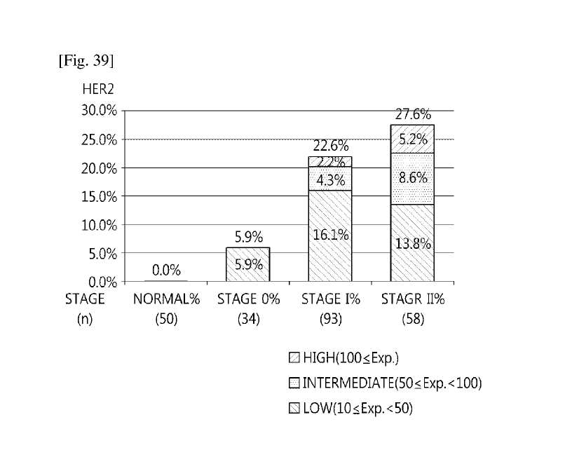

In FIGS. 37 to 39, in the case of the cancer-related intracellular marker, it was verified that there was a difference in expression rate for each stage of the breast cancer patient. It was verified that in both the hTERT and the Ki67 including the HER2, as the stage progressed, the expression of the cancer marker in the blood was increased. Particularly, it was verified that a ratio of overexpressed patients of 90 to 100 times or more was increased as the stage of the patient was deteriorated. As a result, it is verified that the intercellular cancer-related marker is associated with the stage of the patient, and thus it is considered that the malignance of the breast cancer of the patient can be verified by a rapid diagnosis using the blood.

1. An information providing method for diagnosing breast cancer, comprising:
a) isolating a total RNA from cells obtained from tissue or blood of a cancer suspected patient;
b) synthesizing cDNA from the isolated total RNA;
c) performing real-time-PCR of the synthesized cDNA by using compositions of a primer set and a probe to amplify a human epidermal growth factor receptor (HER) 2, a primer set and a probe to amplify an estrogen receptor, a primer set and a probe to amplify a progesterone receptor, a primer set and a probe to amplify cytokeratin 19, a primer set and a probe to amplify an epithelial cell adhesion molecule (EpCAM), a primer set and a probe to amplify human telomerase reverse transcriptase (hTERT), a primer set and a probe to amplify Ki67, a primer set and a probe to amplify Vimentin, a primer set and a probe to amplify cyclin D1, a primer set and a probe to amplify E-cadherin (cad), a primer set and a probe to amplify snail, a primer set and a probe to amplify phosphatase and tensin homolog (PTEN), a primer set and a probe to amplify neuroplastin (NPTN), and a primer set and a probe to amplify glyceraldehyde-3-phosphate dehydrogenase (GAPDH); and
d) comparing the amplified level with an expressed level in a normal person.

2. The information providing method for diagnosing the breast cancer of claim 1, wherein the comparing of the amplified level with the amplified level in the normal person is performed by a standard or cut-off value.

3. The information providing method for diagnosing the breast cancer of claim 1, wherein the primer set to amplify the HER 2 may be transcribed in SEQ ID NOS: 1 and 2, 3 and 4, and 6 and 7 and the probe may be transcribed in SEQ ID NOS: 5, 8, and 9, the primer set to amplify the estrogen receptor may be transcribed in SEQ ID NOS: 10 and 11, and 13 and 14 and the probe may be transcribed in SEQ ID NOS: 12 and 15, the primer set to amplify the progesterone receptor may be transcribed in SEQ ID NOS: 16 and 17, and 19 and 20 and the probe may be transcribed in SEQ ID NOS: 18 and 21, the primer set and the probe to amplify the GAPDH are transcribed in SEQ ID NOS: 22 and 23 and 24, respectively, the primer set and the probe to amplify the EpCAM are transcribed in SEQ ID NOS: 25 and 26 and 27, respectively, the primer set and the probe to amplify the cytochrome keratin 19 are transcribed in SEQ ID NOS: 28 and 29 and 30, respectively, the primer set and the probe to amplify the hTERT are transcribed in SEQ ID NOS: 31 and 32 and 33, respectively, the primer set and the probe to amplify the Ki67 are transcribed in SEQ ID NOS: 34 and 35 and 36, respectively, the primer set and the probe to amplify the Vimentin are transcribed in SEQ ID NOS: 37 and 38 and 39, the primer set and the probe to amplify the Cyclin D1 are transcribed in SEQ ID NOS: 40 and 41 and 42, respectively, the primer set and the probe to amplify the E-cad are transcribed in SEQ ID NOS: 43 and 44 and 45, respectively, the primer set and the probe to amplify the Snail are transcribed in SEQ ID NOS: 46 and 47 and 48, respectively, the primer set and the probe to amplify the PTEN are transcribed in SEQ ID NOS: 49 and 50 and 51, respectively, the primer set and the probe to amplify the NPTN are transcribed in SEQ ID NOS: 52 and 53 and 54, respectively.

4. A composition of primer sets and probes for diagnosing breast cancer, comprising:
a primer set to amplify HER 2 transcribed in SEQ ID NOS: 1 and 2, 3 and 4, and 6 and 7 and a probe transcribed in SEQ ID NOS: 5, 8, and 9, a primer set to amplify an estrogen receptor transcribed in SEQ ID NOS: 10 and 11 and 13 and 14 and a probe transcribed in SEQ ID NOS: 12 and 15, a primer set to amplify a progesterone receptor transcribed in SEQ ID NOS: 16 and and 19 and 20 and a probe transcribed in SEQ ID NOS: 18 and 21, a primer set and a probe to amplify GAPDH transcribed in SEQ ID NOS: 22 and 23 and 24, respectively, a primer set and a probe to amplify EpCAM transcribed in SEQ ID NOS: 25, 26 and 27, respectively, a primer set and a probe to amplify cytochrome keratin 19 transcribed in SEQ ID NOS: 28 and 29 and 30, respectively, a primer set and a probe to amplify hTERT transcribed in SEQ ID NOS: 31 and 32 and 33, respectively, a primer set and a probe to amplify Ki67 transcribed in SEQ ID NOS: 34 and 35 and 36, respectively, a primer set and a probe to amplify Vimentin transcribed in SEQ ID NOS: 37 and 38 and 39, a primer set and a probe to amplify Cyclin D1 transcribed in SEQ ID NOS: 40 and 41 and 42, respectively, a primer set and a probe to amplify E-cad transcribed in SEQ ID NOS: 43 and 44 and 45, respectively, a primer set and a probe to amplify Snail transcribed in SEQ ID NOS: 46 and 47 and 48, respectively, a primer set and a probe to amplify PTEN transcribed in SEQ ID NOS: 49 and 50 and 51, respectively, and a primer set and a probe to amplify NPTN transcribed in SEQ ID NOS: 52 and 53 and 54, respectively.

5. The composition of primer sets and probes for diagnosing breast cancer of claim 4, wherein a 5′-terminal of the probe is labeled with a fluorescent material.

6. (canceled)

7. A kit for diagnosing breast cancer comprising the composition of claim 4.

8. A kit for a diagnosis of breast cancer in an early stage or for each stage including the composition of claim 4.


SEQUENCE LISTING (54 sequences)

SEQ ID NO: 1 - DNA, 26 bp, Artificial Sequence
  primer
aacctggaactcacctacctgcccac

SEQ ID NO: 2 - DNA, 22 bp, Artificial Sequence
  primer
cgatgagcacgtagccctgcac

SEQ ID NO: 3 - DNA, 23 bp, Artificial Sequence
  primer
aactcacctacctgcccaccaat

SEQ ID NO: 4 - DNA, 20 bp, Artificial Sequence
  primer
cacgtagccctgcacctcct

SEQ ID NO: 5 - DNA, 25 bp, Artificial Sequence
  probe
cagcctgtccttcctgcaggatatc

SEQ ID NO: 6 - DNA, 22 bp, Artificial Sequence
  primer
aagcatacgtgatggctggtgt

SEQ ID NO: 7 - DNA, 24 bp, Artificial Sequence
  primer
tctaagaggcagccatagggcata

SEQ ID NO: 8 - DNA, 26 bp, Artificial Sequence
  probe
atatgtctcccgccttctgggcatct

SEQ ID NO: 9 - DNA, 25 bp, Artificial Sequence
  probe
catccacggtgcagctggtgacaca

SEQ ID NO: 10 - DNA, 26 bp, Artificial Sequence
  primer
tcaaatgccaaattgtgtttgatgga

SEQ ID NO: 11 - DNA, 25 bp, Artificial Sequence
  primer
gctgcgacaaaaccgagtcacatca

SEQ ID NO: 12 - DNA, 27 bp, Artificial Sequence
  probe
taatatgcccttttgccgatgcatact

SEQ ID NO: 13 - DNA, 23 bp, Artificial Sequence
  primer
atgccaaattgtgtttgatggat

SEQ ID NO: 14 - DNA, 26 bp, Artificial Sequence
  primer
cgacaaaaccgagtcacatcagtaat

SEQ ID NO: 15 - DNA, 19 bp, Artificial Sequence
  probe
atgcccttttgccgatgca

SEQ ID NO: 16 - DNA, 24 bp, Artificial Sequence
  primer
agtcagagttgtgagagcactgga

SEQ ID NO: 17 - DNA, 24 bp, Artificial Sequence
  primer
ctggcttagggcttggctttcatt

SEQ ID NO: 18 - DNA, 24 bp, Artificial Sequence
  probe
tgctgttgctctcccacagccagt

SEQ ID NO: 19 - DNA, 24 bp, Artificial Sequence
  primer
gttgtgagagcactggatgctgtt

SEQ ID NO: 20 - DNA, 24 bp, Artificial Sequence
  primer
aatctctggcttagggcttggctt

SEQ ID NO: 21 - DNA, 24 bp, Artificial Sequence
  probe
acagccagtgggcgttccaaatga

SEQ ID NO: 22 - DNA, 22 bp, Artificial Sequence
  primer
ccatcttccaggagcgagatcc

SEQ ID NO: 23 - DNA, 21 bp, Artificial Sequence
  primer
atggtggtgaagacgccagtg

SEQ ID NO: 24 - DNA, 24 bp, Artificial Sequence
  probe
tccacgacgtactcagcgccagca

SEQ ID NO: 25 - DNA, 24 bp, Artificial Sequence
  primer
gccagtgtacttcagttggtgcac

SEQ ID NO: 26 - DNA, 24 bp, Artificial Sequence
  primer
catttctgccttcatcaccaaaca

SEQ ID NO: 27 - DNA, 29 bp, Artificial Sequence
  probe
tactgtcatttgctcaaagctggctgcca

SEQ ID NO: 28 - DNA, 20 bp, Artificial Sequence
  primer
gatgagcaggtccgaggtta

SEQ ID NO: 29 - DNA, 20 bp, Artificial Sequence
  primer
tcttccaaggcagctttcat

SEQ ID NO: 30 - DNA, 23 bp, Artificial Sequence
  probe
ctgcggcgcacccttcagggtct

SEQ ID NO: 31 - DNA, 22 bp, Artificial Sequence
  primer
tgacgtccagactccgcttcat

SEQ ID NO: 32 - DNA, 22 bp, Artificial Sequence
  primer
acgttctggctcccacgacgta

SEQ ID NO: 33 - DNA, 24 bp, Artificial Sequence
  probe
gctgcggccgattgtgaacatgga

SEQ ID NO: 34 - DNA, 25 bp, Artificial Sequence
  primer
taatgagagtgagggaatacctttg

SEQ ID NO: 35 - DNA, 25 bp, Artificial Sequence
  primer
aggcaagttttcatcaaatagttca

SEQ ID NO: 36 - DNA, 22 bp, Artificial Sequence
  probe
ggcgtgtgtcctttggtgggca

SEQ ID NO: 37 - DNA, 23 bp, Artificial Sequence
  primer
atgttgacaatgcgtctctggca

SEQ ID NO: 38 - DNA, 27 bp, Artificial Sequence
  primer
atttcctcttcgtggagtttcttcaaa

SEQ ID NO: 39 - DNA, 29 bp, Artificial Sequence
  probe
tgaccttgaacgcaaagtggaatctttgc

SEQ ID NO: 40 - DNA, 24 bp, Artificial Sequence
  primer
tgaacaagctcaagtggaacctgg

SEQ ID NO: 41 - DNA, 24 bp, Artificial Sequence
  primer
ctgtttgttctcctccgcctctgg

SEQ ID NO: 42 - DNA, 26 bp, Artificial Sequence
  probe
cccgcacgatttcattgaacacttcc

SEQ ID NO: 43 - DNA, 25 bp, Artificial Sequence
  primer
gggcacagatggtgtgattacagtc

SEQ ID NO: 44 - DNA, 22 bp, Artificial Sequence
  primer
cccaggcgtagaccaagaaatg

SEQ ID NO: 45 - DNA, 29 bp, Artificial Sequence
  probe
ggcctctacggtttcataacccacagatc

SEQ ID NO: 46 - DNA, 25 bp, Artificial Sequence
  primer
tgtgacaaggaatatgtgagcctgg

SEQ ID NO: 47 - DNA, 22 bp, Artificial Sequence
  primer
cgcagatcttgcaaacacaagg

SEQ ID NO: 48 - DNA, 30 bp, Artificial Sequence
  probe
cctgaagatgcatattcggacccacacatt

SEQ ID NO: 49 - DNA, 23 bp, Artificial Sequence
  primer
gtctgagtcgcctgtcaccattt

SEQ ID NO: 50 - DNA, 20 bp, Artificial Sequence
  primer
cgccgtgttggaggcagtag

SEQ ID NO: 51 - DNA, 25 bp, Artificial Sequence
  probe
tgggaacgccggagagttggtctct

SEQ ID NO: 52 - DNA, 28 bp, Artificial Sequence
  primer
accagtgaagaggtcattattcgagaca

SEQ ID NO: 53 - DNA, 26 bp, Artificial Sequence
  primer
tatgtaagggtgtgagagctggaggt

SEQ ID NO: 54 - DNA, 30 bp, Artificial Sequence
  probe
cctgttctccctgtcaccctgcagtgtaac